(19) United States
(12) Patent Application Publication
(10) Pub. No.: US 2005/0272752 A1
(43) Pub. Date: Dec. 8, 2005

(54) Complexes of E-2-Methoxy-N-(3-{4-[3-methyl-4-(6-methyl-pyridin-3-yloxy)-phenylamino]-quinazolin-6-yl}-allyl)-acetamide and methods of preparing same

(75) Inventors: Phillip James Johnson, North Stonington, CT (US); David Harold Brown Ripin, Old Saybrook, CT (US); Peter Robert Rose, Ledyard, CT (US); Jeanene Elizabeth Tickner, Waterford, CT (US); Michael Girard Vetelino, North Stonington, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/136,775

(22) Filed: May 25, 2005

Related U.S. Application Data
(60) Provisional application No. 60/578,001, filed on Jun. 7, 2004.

(51) Int. Cl. A61K 31/517

(52) U.S. Cl. 514/266.21; 544/284

(57) ABSTRACT

This invention relates to methods of making sesquisuccinate complexes of E-2-Methoxy-N-(3{4-[3-methyl-4-(6-methyl-pyridin-3-yloxy)-phenylamino]-quinazolin-6-yl}-allyl)-acetamide.

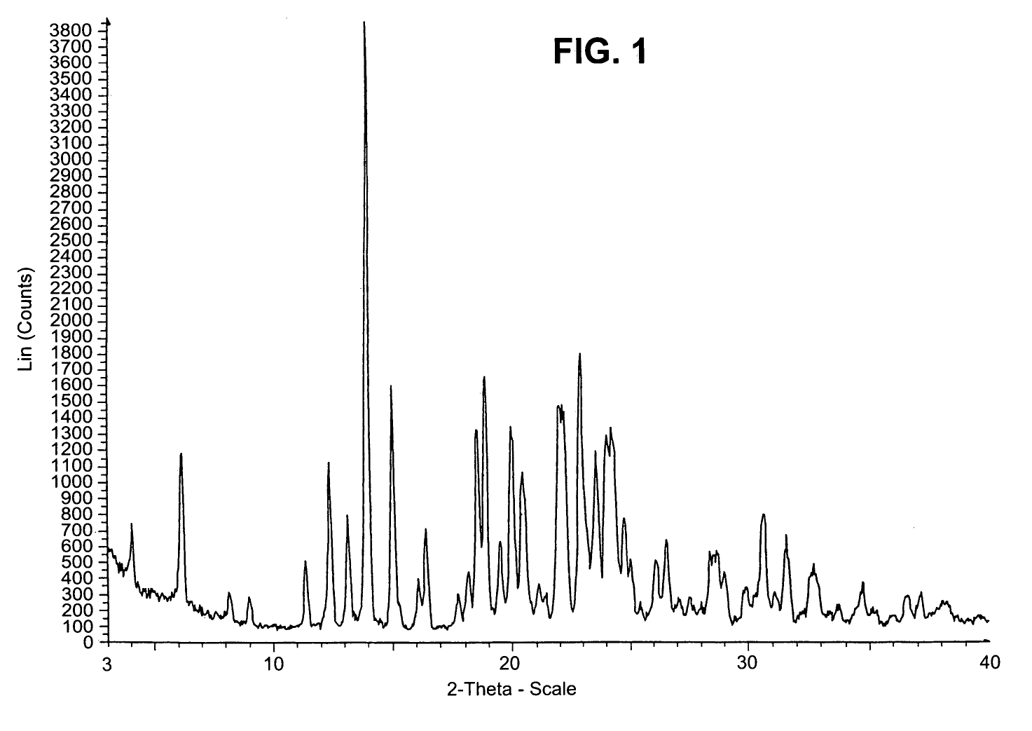

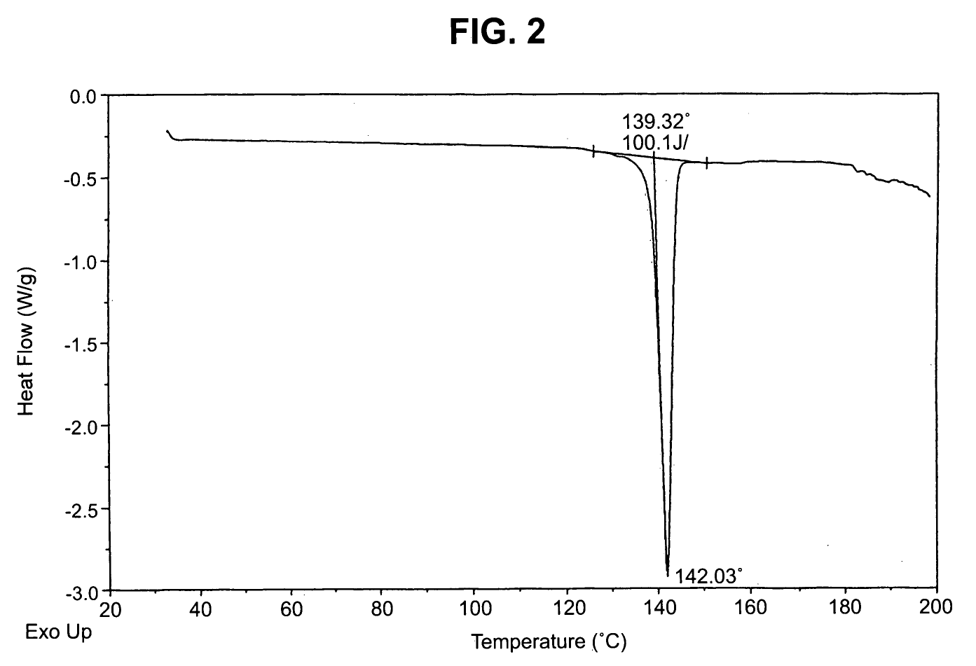

### CROSS-REFERENCE TO RELATED APPLICATION(S)

Reference is made to U.S. application Ser. No. 60/578,001, filed Jun. 7, 2004, the disclosure of which is incorporated herein by reference in its entirety.

### BACKGROUND OF THE INVENTION

This invention relates to methods of making sesquisuccinate complexes of E-2-Methoxy-N-(3-{4-[3-methyl-4-(6-methyl-pyridin-3-yloxy)-phenylamino]-quinazolin-6-yl}-ally)-acetamide having the formula I:

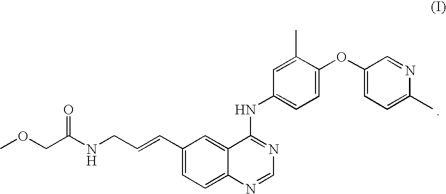

Formula I in its free base form is described in co-pending U.S. Ser. No. 09/883,752, filed Jun. 18, 2001. In addition, the succinate and malonate complexes of E-2-Methoxy-N-(3-{4-[3-methyl-4-(6-methyl-pyridin-3-yloxy)-phenylamino]-quinazolin-6-yl}-allyl)-acetamide are described in copending U.S. application Ser. No. 10/315,862, filed Dec. 12, 2002, and other complexes of E-2-Methoxy-N-(3-{4-[3-methyl-4-(6-methyl-pyridin-3-yloxy)-phenylamino]-quinazolin-6-yl}-allyl)-acetamide are described in copending U.S. application Ser. No. 60/434,700, filed Dec. 19, 2002. Each of the applications identified above are assigned in common with the present application and their respective disclosures are incorporated herein by reference in their entireties.

Still further, reference is also made to U.S. Pat. Nos. 6,284,764; 6,541,481; 6,465,449; WO 01/98277, filed Jun. 14, 2001; WO 03/050108, filed Nov. 11, 2002; U.S. application Ser. Nos. 10/121,831, filed Apr. 13, 2001; 10/315,862, filed Nov. 11, 2002; 10/737,691, filed Dec. 8, 2003; 60/548,743, filed Feb. 27, 2004; and 60/434,700, filed Dec. 8, 2003. The disclosures of each of these issued patents, PCT publications, and pending applications are incorporated herein by reference in their entireties.

It has been recognized that inhibitors of receptor tyrosine kinases are useful as selective inhibitors of the growth of mammalian cancer cells. For example, erbstatin, a tyrosine kinase inhibitor, selectively attenuates the growth in athymic nude mice of a transplanted human mammary carcinoma which expresses epidermal growth factor receptor tyrosine kinase (EGFR) but is without effect on the growth of another carcinoma which does not express the EGF receptor. Thus, the compounds of the present invention, which are selective inhibitors of certain receptor tyrosine kinases, are useful in the treatment of abnormal cell growth, in particular cancer, in mammals. In addition to receptor tyrosine kinases, the compounds of the present invention can also display inhibitory activity against a variety of other non-receptor tyrosine kinases (eg: Ick, src, abl) or serine/threonine kinases (e.g.: cyclin dependent kinases).

Various other compounds, such as styrene derivatives, have also been shown to possess tyrosine kinase inhibitory properties. More recently, five European patent publications, namely EP 0 566 226 A1 (published Oct. 20, 1993), EP 0 602 851 A1 (published Jun. 22, 1994), EP 0 635 507 A1 (published Jan. 25, 1995), EP 0 635 498 A1 (published Jan. 25, 1995), and EP 0 520 722 A1 (published Dec. 30, 1992), refer to certain bicyclic derivatives, in particular quinazoline derivatives, as possessing anti-cancer properties that result from their tyrosine kinase inhibitory properties. Also, World Patent Application WO 92/20642 (published Nov. 26, 1992), refers to certain bis-mono and bicyclic aryl and heteroaryl compounds as tyrosine kinase inhibitors that are useful in inhibiting abnormal cell proliferation. World Patent Applications WO96/16960 (published Jun. 6, 1996), WO 96/09294 (published Mar. 6, 1996), WO 97/30034 (published Aug. 21, 1997), WO 98/02434 (published Jan. 22, 1998), WO 98/02437 (published Jan. 22, 1998), and WO 98/02438 (published Jan. 22, 1998), also refer to substituted bicyclic heteroaromatic derivatives as tyrosine kinase inhibitors that are useful for the same purpose. Other patent applications that refer to anti-cancer compounds are U.S. patent application Ser. Nos. 09/488,350 (filed Jan. 20, 2000) and 09/488,378 (filed Jan. 20, 2000), both of which are incorporated herein by reference in their entirety.

While the sesquisuccinate complex was synthesized by the method outlined in U.S. application Ser. No. 60/434,700, an optimized method for preparing the sesquisuccinate complex is desirable, especially one that involves a reproducible crystallization process, with optimal yield, and one which can be scaled to commercial synthesis. Therefore, a need exists for an optimized method of preparing the sesquisuccinate complex of E-2-Methoxy-N-(3-{4-[3-methyl-4-(6-methyl-pyridin-3-yloxy)-phenylamino]-quinazolin-6-yl}-allyl)-acetamide.

### SUMMARY OF THE INVENTION

The present invention provides a method of making a sesquisuccinate complex of E-2-Methoxy-N-(3-{4-[3-methyl-4-(6-methyl-pyridin-3-yloxy)-phenylamino]-quinazolin-6-yl}-acetamide by a method comprising:

(a) charging a reaction vessel with (i) the free base of E-2-Methoxy-N-(3-{4-[3-methyl-4-(6-methyl-pyridin-3-yloxy)-phenylamino]-quinazolin-6-yl}-allyl)-acetamide, (ii) a reaction solvent selected from the group consisting of acetone, aqueous acetone (containing up to 15% (v:v) water in acetone), acetonitrile, tetrahydrofuran, 2-propanol, 1-butanol, 1-propanol, 2-methyl-tetrahydrofuran, 3-methyl-1-butanol, and mixtures thereof, and (iii) a molar excess of succinic acid; and

(b) heating the mixture formed (a) for a time and at a temperature sufficient to allow formation of the sesquisuccinate complex of E-2-Methoxy-N-(3-{4-[3-methyl4-(6-methyl-pyridin-3-yloxy)-phenylamino]-quinazolin-6-yl}-allyl)-acetamide (hereafter referred to as the “sesquisuccinate complex”).

The method of the present invention further comprises one or more of the following steps

(c) isolating the sesquisuccinate complex at a temperature that is compatible with complex stability;

(d) washing said sesquisuccinate complex with a reaction solvent; and/or

(e) drying said sesquisuccinate complex.

Another embodiment of the present invention provides a reaction solvent selected from the group consisting of acetone, aqueous acetone, 1-propanol, 2-propanol, 1-butanol, acetonitrile, tetrahydrofuran, 2-methyl tetrahydrofuran, 3-methyl-1-butanol, and mixtures thereof. In a preferred embodiment, the reaction solvent is selected from the group consisting of acetone, aqueous acetone, tetrahydrofuran, acetonitrile, 2-propanol, and mixtures thereof.

In a preferred embodiment, between about 10 ml/g to 30 ml/g reaction solvent is added in step (a) of the method of the present invention.

In another embodiment, the reaction solvent is aqueous acetone and its comprised of up to 15% water (v:v) in acetone, preferably between about 5 and 15% water (v:v) in acetone, and most preferably up to 10% water (v:v) in acetone.

Still further, the invention provides that a molar excess of succinic acid is added in step (a). The skilled artisan will readily appreciate that this amount of succinic acid refers to the number of equivalents of succinic acid in the reaction medium in comparison to the amount (in moles) of free base. Thus, a “molar excess” of succinic acid refers to greater than one molar equivalent of succinic acid. In a preferred embodiment, the amount of succinic acid added is in the range of 1.5 to 3.5 equivalents; preferably 1.5 to 3 equivalents, and most preferably 2 to 3 equivalents.

The method of the present invention contemplates that the mixture formed in step (a) is heated to a temperature that is sufficient to dissolve the reaction mixture. In a preferred embodiment, the mixture formed in (a) is heated to a temperature sufficient to dissolve the sesquisuccinate complex.

Still further, the present invention provides that the mixture formed in (a) should be stirred for a time and at a temperature that is sufficient to allow complex formation. In a preferred embodiment, the mixture is heated for at least 1 hour, and preferably between 1 and 24 hours.

Another embodiment of the present invention is a method further comprising (b)(i) maintaining the mixture formed in step (a) for a time and at a temperature sufficient to allow crystallization to occur. For example, the reaction mixture formed in step (a) is stirred for at least 1 hour. Still further, the method may also include the step of (b)(ii) cooling the mixture formed in (b)(i) for a time and at a temperature sufficient to facilitate crystal growth, e.g., at least 1 hour. The skilled artisan will readily appreciate that the times and temperatures required for this step are flexible, depending on many factors, especially the choice of solvent, ambient humidity, etc. However, such conditions may be modified by the skilled artisan to facilitate crystal growth.

In a further embodiment, the method also comprises seeding the mixture formed in step (a) with sesquisuccinate complex to facilitate crystal growth. For example, up to 1% w/w sesquisuccinate complex is added to facilitate crystal growth. Seeding is optional, but it may aid in the robustness of crystallization and increase the speed of the crystallization process.

In a further embodiment, the method also comprises cooling of the product slurry formed in step (b)(i) at a cooling rate sufficient to achieve a productive or optimized yield. The skilled artisan will readily understand that the cooling may be performed using a variety of cooling trajectories, including but not limited to linear cooling ramps, parabolic cooling ramps, and cooling-heating cycling (also known as Ostwald Ripening, i.e., the growth of larger crystals from those of smaller size which have a higher solubility than the larger crystals). Methods of crystallization of organic compounds and pharmaceuticals are known, or will be apparent, to those skilled in the art. For examples, see Mullin's Crystallization, Butterworth-Heinemann, Oxford, UK 3d Ed. (1997), which is incorporated herein by reference.

The method of the present invention optionally includes isolating the sesquisuccinate complex at a temperature that is compatible with complex stability. The skilled artisan will readily understand that this temperature is a range not exceeding the melting point of the sesquisuccinate complex (approximately 139° C.). In a preferred embodiment, the sesquisuccinate complex is isolated at about 0 to about 20° C.

It has unexpectedly been found that the sesquisuccinate complex of E-2-Methoxy-N-(3-{4-[3-methyl4-(6-methyl-pyridin-3-yloxy)-phenylamino]-quinazolin-6-yl}-allyl)-acetamide is highly crystalline, i.e., substantially free of amorphous material. Such complexes have the advantage that they provide more reproducible dosing results. The sesquisuccinate complex of E-2-Methoxy-N-(3-{4-[3-methyl-4-(6-methyl-pyridin-3-yloxy)-phenylamino]-quinazolin-6-yl}-allyl)-acetamide is also substantially hygroscopically stable, which alleviates potential problems associated with weight changes of the active ingredient during the manufacture of capsules or tablets.

Therefore, the synthetic method of the present invention may be used to prepare the sesquisuccinate complex of E-2-Methoxy-N-(3-{4-[3-methyl-4-(6-methyl-pyridin-3-yloxy)-phenylamino]-quinazolin-6-yl}-allyl)-acetamide, which is useful in a method for the treatment of abnormal cell growth in a mammal, including a human, comprising administering to said mammal an amount of a sesquisuccinate complex of E-2-Methoxy-N-(3-{4-[3-methyl-4-(6-methyl-pyridin-3-yloxy)-phenylamino]-quinazolin-6-yl}-allyl)-acetamide that is effective in treating abnormal cell growth. In one embodiment of this method, the abnormal cell growth is cancer, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In another embodiment of said method, said abnormal cell growth is a benign proliferative disease, including, but not limited to, psoriasis, benign prostatic hypertrophy or restinosis.

This invention also relates to a method for the treatment of abnormal cell growth in a mammal which comprises administering to said mammal an amount of the sesquisuccinate complex of E-2-Methoxy-N-(3-{4-[3-methyl-4-(6-methyl-pyridin-3-yloxy)-phenylamino]-quinazolin-6-yl}-allyl)-acetamide prepared by the method of the present invention, that is effective in treating abnormal cell growth in combination with an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens.

The method of the present invention may therefore be used to prepare the sesquisuccinate complex of E-2-Methoxy-N-(3-{4-[3-methyl4-(6-methyl-pyridin-3-yloxy)-phenylamino]-quinazolin-6-yl}-allyl)-acetamide, which may be used alone or in combination with other anti-tumor or paliative agents in a pharmaceutical composition.

Further, the free base used to prepare the sesquisuccinate complex of the present invention exists in various crystal forms and/or in an amorphous form. The crystal forms of the free base include Form A and hydrates and/or solvates thereof; Form B and hydrates and/or solvates thereof; Form C and hydrates and/or solvates thereof; Form F and hydrates and/or solvates thereof; Form G and hydrates and/or solvates thereof, and Form H and hydrates and/or solvates thereof. In a preferred embodiment, the free base exists in crystal form A. These crystal forms are described fully in copending U.S. application Ser. No. 60/548,743, filed Feb. 27, 2004, which is incorporated herein by reference in its entirety. Moreover, those skilled in the art are aware that other forms may be synthesized by modifications to the synthetic methods employed in preparing the crystal forms disclosed herein and in the copending application, U.S. Ser. No. 60/548,743.

The subject invention also includes isotopically-labeled compounds, and the sesquisuccinate complex which are identical to the sesquisuccinate complex, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F, and ³⁶Cl, respectively. Sesquisuccinate complexes which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled sesquisuccinate complexes of this invention can generally be prepared by carrying out the procedures disclosed in the Schemes and/or in the Examples and Preparations below, by substituting a readily available isotopically labelled reagent for a non-isotopically labelled reagent.

### DETAILED DESCRIPTION OF THE INVENTION

General synthetic methods which may be referred to for preparing the compounds of the present invention are provided in U.S. Pat. No. 5,747,498 (issued May 5, 1998), U.S. patent application Ser. No. 08/953078 (filed Oct. 17, 1997), WO 98/02434 (published Jan. 22, 1998), WO 98/02438 (published Jan. 22, 1998), WO 96/40142 (published Dec. 19, 1996), WO 96/09294 (published Mar. 6, 1996), WO 97/03069 (published Jan. 30, 1997), WO 95/19774 (published July 27, 1995) and WO 97/13771 (published Apr. 17, 1997). Additional procedures are referred to in U.S. patent application Ser. No. 09/488,350 (filed Jan. 20, 2000) and 09/488,378 (filed Jan. 20, 2000). The foregoing patents and patent applications are incorporated herein by reference in their entirety. Certain starting materials may be prepared according to methods familiar to those skilled in the art and certain synthetic modifications may be done according to methods familiar to those skilled in the art. A standard procedure for preparing 6-iodoquinazolinone is provided in Stevenson, T. M., Kazmierczak, F., Leonard, N. J., J. Org. Chem., 1986, 51, 5, p. 616. Palladium-catalyzed boronic acid couplings are described in Miyaura, N., Yanagi, T., Suzuki, A. Syn. Comm., 1981, 11, 7, p. 513. Palladium catalyzed Heck couplings are described in Heck et. al., Organic Reactions, 1982, 27, p. 345 or Cabri et. al. in Acc. Chem. Res., 1995, 28, 2. For examples of the palladium catalyzed coupling of terminal alkynes to aryl halides see: Castro et. al., J. Org. Chem., 1963, 28, 3136. or Sonogashira et. al., Synthesis, 1977, 777. Terminal alkyne synthesis may be performed using appropriately substituted/protected aldehydes as described in: Colvin, E. W. J. et. al., Chem. Soc. Perkin Trans. I, 1977, 869; Gilbert, J. C. et. al., J. Org. Chem., 47, 10, 1982; Hauske, J. R. et. al., Tet. Lett., 33, 26, 1992, 3715; Ohira, S. et. al., J. Chem. Soc. Chem. Commun., 9, 1992, 721; Trost, B. M. J., Amer. Chem. Soc., 119, 4,1997, 698; or Marshall, J. A. et. al., J. Org. Chem., 62, 13, 1997, 4313.

Alternatively terminal alkynes may be prepared by a two step procedure. First, the addition of the lithium anion of TMS (trimethylsilyl) acetylene to an appropriately substituted/protected aldehyde as in: Nakatani, K. et. al., Tetrahedron, 49, 9, 1993, 1901. Subsequent deprotection by base may then be used to isolate the intermediate terminal alkyne as in Malacria, M.; Tetrahedron, 33, 1977, 2813; or White, J. D. et. al., Tet. Lett., 31, 1, 1990, 59.

In a preferred embodiment, the synthetic method of preparing the free base of E-2-Methoxy-N-(3-{4-[3-methyl4-(6-methyl-pyridin-3-yloxy)-phenylamino]-quinazolin-6-yl}-allyl)-acetamide is that disclosed in U.S. application Publication No. 20030144506, filed Dec. 2, 2002, as well as WO 03/045939, filed Oct. 3, 2002. Each of these publications are incorporated by reference herein in their entireties.

In one embodiment of the present invention, the sesquisuccinate complex may be prepared by the following method:

(a) A reaction vessel is charged with the E-2-Methoxy-N-(3-{4-[3-methyl4-(6-methyl-pyridin-3-yloxy)-phenylamino]-quinazolin-6-yl}-allyl)-acetamide free base (“free base”) (1 equivalent) and solvent (10 mL/g to about 30 mL/g). Due to variations in solubility, some solvent/free base reaction mixtures require more solvent than others to produce an easily stirrable slurry, but the amount of solvent required to produce such a slurry is generally about 10 mL/g to about 30 mL/g. In a preferred embodiment, the solvent is selected from the group consisting of acetone, aqueous acetone, acetonitrile, tetrahydrofuran, 2-propanol, and mixtures thereof. In an alternative embodiment, the solvent is selected from the group consisting of 1-butanol, 1-propanol, 2-methyl tetrahydrofuran, 3-methyl-i-butanol, and mixtures thereof.

(b) Succinic acid (1.6 to 3 equivalents; Sigma-Aldrich Corp. St. Louis, Mo., USA) is added to the reaction vessel. In a preferred embodiment, approximately 2 equivalents of succinic acid per equivalent of free base is added to the reaction mixture.

(c) The mixture is heated to about 50 to about 60° C. to allow solution to occur. Generally, the temperature of heating varies according to the solvent in the mixture. Temperatures up to and including reflux are suitable.

(d) Stir the reaction mixture at a temperature and for a suitable time to allow crystallization to begin (typically at least one hour). Crystallization may not take place at highly elevated temperatures due to the solubility of the crystals in the reaction solvent. Typical temperatures of about 40 to 50° C. in 10-20 mL/g solvent are sufficient to initiate crystallization. Crystallization may be facilitated by seeding the reaction mixture with a small amount of sesquisuccinate complex. Seeding is not absolutely necessary but it faciliates crystallization/nucleation. The solution or slurry is cooled (over at least 1 hour, and preferably between about 2 and 8 hours). A longer cooling time facilitates crystal growth, but it is not necessary to yield the correct crystalline form. A variety of cooling profiles may be used, including linear cooling profiles, parabolic cooling profiles, and cooling-heating cycling profiles to modify the particle size, but these are not necessary to achieve the correct crystalline form or optimal yields.

(e) Isolate crystals at about 0 to about 20° C.

(f) Wash product with succinic acid soluble solvent. Solvents shown to be successful for this are acetone, 2-propanol, acetonitrile, tetrahydrofuran, and mixtures thereof. The solvent wash of the cake is critical to remove excess succinic acid.

(g) Dry product to a constant weight to remove solvent. Drying has been completed by air drying and vacuum drying in vacuum ovens ranging from 20 to 60° C.

In each of the reactions discussed or illustrated in the Schemes above, pressure is not critical unless otherwise indicated. Pressures from about 0.5 atmospheres to about 5 atmospheres are generally acceptable, and ambient pressure, i.e., about 1 atmosphere, is preferred as a matter of convenience.

The complexes of the present invention are useful in the treatment of hyperproliferative diseases, such as cancers, in mammals, especially humans, and to pharmaceutical compositions containing such compounds.

The sesquisuccinate complex of the present invention has been characterized using elemental analysis. Further, the in vitro activity of the sesquisuccinate complex may be determined by the following procedure.

The in vitro activity of the sesquisuccinate complex as erbB kinase inhibitors in intact cells may be determined by the following procedure. Cells, for example 3T3 cells transfected with human EGFR (Cohen et al., J. Virology, 67:5303, 1993) or with chimeric EGFR/erbB2 kinase (EGFR extracellular/erbB2 intracellular, Fazioli et al., Mol. Cell. Biol., 11: 2040, 1991) are plated in 96-well plates at 12,000 cells per well in 100 pi medium (Dulbecco's Minimum Essential Medium (DMEM) with 5% fetal calf serum, 1% pen/streptomycin, 1% L-glutamine) and incubated at 37° C., 5% CO₂ Test compounds are solubilized in DMSO at a concentration of 10 mM, and tested at final concentrations of 0, 0.3 μM, 1 μM, 0.3 μM, 0.1 μM and 10 μM in the medium. The cells are incubated at 37° C. for 2 hours. EGF (40 ng/ml final) is added to each well and cells incubate at room temperature for 15 minutes followed by aspiration of medium, then 100 μl/well cold fixative (50% ethanol/50% acetone containing 200 micromolar sodium orthovanadate) is added. The plate is incubated for 30 minutes at room temperature followed by washing with wash buffer (0.5% Tween 20 in phosphate buffered saline). Blocking buffer (3% bovine serum albumin, 0.05% Tween 20, 200 μM sodium orthovanadate in phosphate buffered saline, 100 μl/well) is added followed by incubation for 2 hours at room temperature followed by two washes with wash buffer. PY54 monoclonal anti-phosphotyrosine antibody directly conjugated to horseradish peroxidase (50 μl/well, 1 μg/ml in blocking buffer) or blocked conjugate (1 μg/ml with 1 mM phosphotyrosine in blocking buffer, to check specificity) is added and the plates incubated for 2 hours at room temperature. The plate wells are then washed 4 times with wash buffer. The colorimetric signal is developed by addition of TMB Microwell Peroxidase Substrate (Kirkegaard and Perry, Gaithersburg, Md.), 50 μl per well, and stopped by the addition of 0.09 M sulfuric acid, 50 μl per well. Absorbance at 450 nM represents phosphotyrosine content of proteins. The increase in signal in EGF-treated cells over control (non-EGF treated) represents the activity of the EGFR or EGFR/chimera respectively. The potency of an inhibitor is determined by measurement of the concentration of compound needed to inhibit the increase in phosphotyrosine by 50% (IC₅₀) in each cell line. The selectivity of the compounds for erbB2 vs. EGFR is determined by comparison of the IC₅₀ for the EGFR transfectant vs. that for the erbB2/EGFR chimera transfectant. Thus, for example, a compound with an IC₅₀ of 100 nM for the EGFR transfectant and 10 nM for the erbB2/EGFR chimera transfectant is considered 10-fold selective for erbB2 kinase.

Administration of the sesquisuccinate complex can be effected by any method that enables delivery of the compounds to the site of action. These methods include oral routes, intraduodenal routes, parenteral injection (including intravenous, subcutaneous, intramuscular, intravascular or infusion), topical, and rectal administration.

The amount of the sesquisuccinate complex administered will be dependent on the subject being treated, the severity of the disorder or condition, the rate of administration and the judgement of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.2 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

The sesquisuccinate complex may be applied as a sole therapy or may involve one or more other anti-tumor substances, such as those identified hereinabove. Such conjoint treatment may be achieved by way of the simultaneous, sequential or separate dosing of the individual components of the treatment.

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

In the following examples and preparations, “Et” means ethyl, “AC” means acetyl, “Me” means methyl, “ETOAC” or “ETOAc” means ethyl acetate, “THF” means tetrahydrofuran, and “Bu” means butyl.

### EXAMPLE 1

A suitable clean, dry reaction vessel equipped with programmable linear temperature control system was charged with acetone (130 ml), water (14 ml), succinic acid (7.55 g, 3 eq.), and E-2-Methoxy-N-(3-{4-[3-methyl4-(6-methyl-pyridin-3-yloxy)-phenylamino]-quinazolin-6-yl}-allyl)-acetamide free base (10.0 g, 1 equivalent). The reaction mixture was heated to about 45-58° C. to yield a solution. Once the reaction mixture dissolved in solution, the temperature was adjusted to about 45° C. The solution was vacuum filtered into a suitable clean, dry, speck and fiber-free crystallization vessel. The crystallization vessel was maintained at a jacket temperature of about 50° C. in order to maintain the reaction vessel temperature at about 45° C. The reaction vessel was rinsed with about 20 ml acetone and pressure rinsed through the filter into the crystallization vessel. The reaction mixture was stirred and the temperature of the reaction vessel was adjusted to about 42-45° C. The reaction vessel was seeded with about 1% w/w sesquisuccinate complex. After initiating crystallization by seeding, the reaction mixture was stirred at about 35-45° C. for at least about 1 hour. The vessel was slowly cooled to about 5-20° C., preferably over about 4 hours. The reaction mixture was stirred at about 5-20° C. for about 18 hours. The sesquisuccinate complex was isolated by filtration on a BUchner style funnel, and the cake was washed with acetone at about 20° C. The sesquisuccinate complex was dried to a constant weight by air-drying or in a vacuum oven ranging from about 20-60° C. Sesquisuccinate complex yield: 85-90% w/w.

### EXAMPLE 2

To a 500 gallon reactor, E-2-Methoxy-N-(3-{4-[3-methyl-4-(6-methyl-pyridin-3-yloxy)-phenylamino]-quinazolin-6-yl}-allyl)acetamide free base (32.1 kg), succinic acid (24.2 kg), Water (44.9 L), and Acetone (417 L) were added. The mixture was heated to 50° C. and held for 1 hr at 50° C. The solution was filtered to render is speck and fiber free. The solution was collected in second 500 gallon reactor held at about 50° C. The first reactor was rinsed with Acetone (64.2 L) and filtered forward to the speck and fiber free reactor. The solution was cooled to about 40-45° C. over about 30 minutes. Seed crystals of the sesquisuccinate complex (321 g) were added at about 40-45° C. The slurry was held for 2 hours at about 40° C. The slurry was then cooled over about 1 hour to 20° C. and held for about 30 minutes. This was followed by heating back to about 40° C. over one half hour and holding at 40° C. The slurry was cooled over 3 hours to about 35° C., followed by cooling over 2 hours to about 30° C., followed by cooling over 1 hour to 25° C. The slurry was then cooled over about 4 hours to about 0° C. and held at 0° C. for 1 hour. The product was isolated by filtration on a pressurized plate filter covered with a suitable cloth filter media. The product solids were washed with acetone (64.2 L). The product solids were dried for 24 hours at 40 to 50° C. to afford 38.0 kg of the sesquisuccinate complex.

### EXAMPLE 3

A 50 mL round bottom flask equipped with a condenser, thermometer, and a magnetic spin bar for agitation was charged with E-2-Methoxy-N-(3-{4-[3-methyl-4-(6-methyl-pyridin-3-yloxy)-phenylamino]-quinazolin-6-yl}-allyl)-acetamide free base (2.0, 1.0 equiv.) and 2-propanol (20 mL). The mixture was heated to solution. A separate 75 mL reactor was charged with succinic acid (0.91 g, 1.8 equivalents) and 2-propanol (10 mL) and warmed to about 45° C. The free base solution was vacuum filtered to remove any solids and was added to the succinic acid solution over about 10 minutes. The resulting slurry was stirred at 45-50° C. for about 1 hour. The slurry was cooled to 20° C. and stirred overnight. The product was isolated by filtration and washed with 2-propanol. The product was dried in a vacuum oven at 30-40° C. for about 6 hours to afford the sesquisuccinate complex (2.34 g, 85% yield).

### EXAMPLE 4

A 50 mL reactor tube equipped with a magnetic spin bar was charged with E-2-Methoxy-N-(3-{4-[3-methyl-4-(6-methyl-pyridin-3-yloxy)-phenylamino]-quinazolin-6-yl}-allyl)-acetamide free base (1.0 g, 1 equivalent) and acetone (10 mL). The mixture was heated to about 50° C. Succinic acid (0.50 g, 2 equivalents) were added to the mixture to give a solution. The mixture was stirred at 50° C. overnight. The resulting slurry was cooled to 20° C. and stirred overnight. The product was isolated by vacuum filtration and the product cake was washed with 5 mL of acetone. The vacuum on the filtration was stopped. The product cake on the filter was covered with 10 mL of acetone and held for about 5 minutes. The wash acetone was then separated from the product cake by vacuum filtration. After the wash solvent was removed from the cake, the vacuum was halted. The product cake on the filter was covered with 10 mL of acetone and held for about 5 minutes. The wash acetone was separated from the product cake by vacuum filtration. The product was dried in a vacuum oven at 40-45° C. overnight to afford 1.05 grams of the sesquisuccinate complex (76% yield).

### EXAMPLE 5

A 50 mL reactor tube equipped with a magnetic spin bar was charged with E-2-Methoxy-N-(3-{4-[3-methyl-4-(6-methyl-pyridin-3-yloxy)-phenylamino]-quinazolin-6-yl}-allyl)-acetamide free base (1.0 g, 1 equivalent) and tetrahydrofuran (10 mL). The mixture was heated to about 50° C. Succinic acid (0.50 g, 2 equivalents) were added to the mixture to give a solution. The mixture was stirred at 50° C. overnight. The resulting slurry was cooled to 20° C. and stirred overnight. The product was isolated by vacuum filtration and the product cake was washed with 5 mL of tetrahydrofuran. The vacuum on the filtration was stopped. The product cake on the filter was covered with 10 mL of tetrahydrofuran and held for about 5 minutes. The wash solvent was separated from the product cake by vacuum filtration. After the wash solvent was removed from the cake, the vacuum was halted. The product cake on the filter was covered with 10 mL of tetrahydrofuan and held for about 5 minutes. The wash solvent was separated from the product cake by vacuum filtration. The product was dried in a vacuum oven at 40-45° C. overnight to afford 1.14 grams of the sesquisuccinate complex (83% yield).

### EXAMPLE 6

A 50 mL reactor tube equipped with a magnetic spin bar was charged with E-2-Methoxy-N-(3{4-[3-methyl4-(6-methyl-pyridin-3-yloxy)-phenylamino]-quinazolin-6-yl{-allyl)-acetamide free base (1.0 g, 1 equivalent) and 1-butanol (15 mL). The mixture was heated to about 50° C. Succinic acid (0.50 g, 2 equivalents) were added to the mixture to give a solution. The mixture was stirred at 50° C. overnight to give a yellow slurry. The slurry was cooled to 25° C. and stirred overnight. The product was isolated by vacuum filtration and the product cake was washed with 5 mL of acetone. The vacuum on the filtration was stopped. The product cake on the filter was covered with 5 mL of 1-butanol and held for about 5 minutes. The wash solvent was then separated from the product cake by vacuum filtration. The product was dried in a vacuum oven at 40-45° C. overnight to afford 1.02 grams of the sesquisuccinate complex in 74% yield.

### EXAMPLE 7

A reactor tube equipped with a magnetic spin bar was charged with E-2-Methoxy-N-(3-{4-[3-methyl-4-(6-methyl-pyridin-3-yloxy)-phenylamino]-quinazolin-6-yl}-allyl)-acetamide free base (2.0 g, 1 equivalent) and acetonitrile (10 mL). The mixture was heated to about 50° C. Succinic acid (1.00 g, 2 equivalents) were added to the mixture. The slurry was stirred at 50° C. overnight. Acetonitrile (5 mL) was added to the slurry and the mixture was stirred at about 50° C. overnight. The slurry was cooled to 20° C. and stirred overnight. The product was isolated by vacuum filtration and the product cake was covered with 5 mL of acetonitrile and held for about 5 minutes. The wash solvent was then separated from the product cake by vacuum filtration. The product was dried in a vacuum oven at 40-45° C. overnight to afford 1.33 grams of the sesquisuccinate complex in 96% yield.

### EXAMPLE 8

A 50 mL round bottom flask with magnetic spin bar was charged with E-2-Methoxy-N-(3-{4-[3-methyl-4-(6-methyl-pyridin-3-yloxy)-phenylamino]-quinazolin-6-yl}-allyl)-acetamide free base (2.0, 1.0 equiv.) and 2-propanol (20 mL). The mixture was heated to solution. A separate 75 mL reactor was charged with succinic acid (0.91 g, 1.8 equivalents) and 2-propanol propanol (10 mL) and warmed to about 45 to 50° C. The free base solution was vacuum filtered to remove any solids and was added to the succinic acid solution over about 10 minutes. The resulting slurry was stirred at 45-50° C. for about 1 hour. The slurry was cooled to 20° C. and stirred overnight. The product was isolated by filtration and washed with 2-propanol. The product was dried in a vacuum oven at 30-40° C. for about 6 hours to afford the sesquisuccinate complex (2.34 g, 85% yield).

### EXAMPLE 9

A suitable clean, dry reaction vessel equipped with programmable linear temperature controls was charged with acetone (130 ml), water (14 ml), succinic acid (7.55 g, 3 eq.), and free base (10.0 g). The amount of succinic acid used in the reaction can range from about 1.5 to 3.5 equivalents. The reaction mixture was stirred at about 45-58° C. to yield a solution. The temperature can be elevated up to the reflux temperature of the solvent to yield a suitable solution. Once the reaction mixture dissolved in solution, the temperature was adjusted to about 45° C. (the temperature should not drop below 42° C.). The solution was filtered in vacuo into a suitable clean, dry, spec-free crystallization vessel. The crystallization vessel was maintained at a jacket temperature of about 50° C. in order to maintain the reaction vessel temperature at about 45° C. The reaction vessel was rinsed with about 20 ml acetone and pressure rinsed through the filter into the crystallization vessel. The reaction mixture was stirred and the temperature of the reaction vessel was adjusted to about 40-45° C. After crystallization begins, the reaction mixture was stirred at about 35-45° C. for at least about 1 hour. The vessel was slowly cooled to about 5-20° C. over about 4 hours. The reaction mixture was stirred at about 20° C. for 18 hours. The sesquisuccinate complex was isolated by filtration on a Buchner funnel, and the cake was washed with acetone. The sesquisuccinate complex was dried to a constant weight using a vacuum oven ranging from about 20-60° C. to afford the sesquisuccinate complex.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

All patents, applications, publications, test methods, literature, and other materials cited herein are hereby incorporated herein by reference in their entireties.

1. A method of making a sesquisuccinate complex of E-2-Methoxy-N-(3-}4-[3-methyl4-(6-methyl-pyridin-3-yloxy)-phenylamino]-quinazolin-6-yl}-allyl)-acetamide having the formula I: 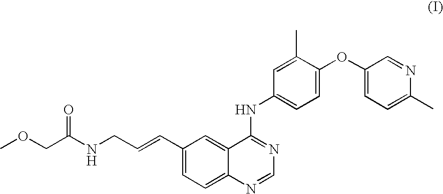
by a method comprising:
(a) charging a reaction vessel with (i) a compound of formula I, (ii) a reaction solvent selected from the group consisting of acetone, aqueous acetone, acetonitrile, tetrahydrofuran, 2-propanol, 1-butanol, 1-propanol, 2-methyl-tetrahydrofuran, 3-methyl-1-butanol, and mixtures thereof, and (iii) a molar excess of succinic acid; and
(b) heating the mixture formed in (a) for a time and at a temperature sufficient to allow formation of said sesquisuccinate complex.

2. The method of claim 1 further comprising
(c) isolating said sesquisuccinate complex at a temperature that is compatible with complex stability.

3. The method of claim 2 further comprising
(d) washing said sesquisuccinate complex isolated in (c) with a reaction solvent.

4. The method of claim 3 further comprising
(e) drying said sesquisuccinate complex washed in (d).

5. The method of claim 1 wherein said reaction solvent is selected from the group consisting of acetone, aqueous acetone, acetonitrile, tetrahydrofuran, 2-propanol, and mixtures thereof.

6. The method of claim 1 wherein 10 ml/g to 30 ml/g reaction solvent is added in step (a).

7. The method of claim 5 wherein said reaction solvent is aqueous acetone that contains up to 15% water (v:v).

8. The method of claim 1 wherein said molar excess of succinic acid is in the range of 1.5 to 3.5 equivalents.

9. The method of claim 8 wherein said range is 1.5 to 3 equivalents.

10. The method of claim 8 wherein said molar excess is 2 equivalents.

11. The method of claim 1 wherein the mixture formed in step (a) is heated up to the reflux temperature of the reaction solvent.

12. The method of claim 11 wherein the mixture is heated to about 50° C.

13. The method of claim 1 further comprising (b)(i) maintaining the mixture formed in step (a) for a time and at a temperature sufficient to allow crystallization to occur.

14. The method of claim 13 wherein said time is at least 1 hour.

15. The method of claim 13 further comprising (b)(ii) cooling the solution formed in (b)(i) for a time and at a temperature sufficient to facilitate crystal growth.

16. The method of claim 15 wherein said time is at least 1 hour.

17. The method of claim 1 further comprising seeding the mixture formed in step (a) with sesquisuccinate complex to facilitate crystal growth.

18. The method of claim 3 wherein said reaction solvent is selected from the group consisting of acetone, 2-propanol, acetonitrile, tetrahydrofuran, and mixtures thereof.

19. The method of claim 17 wherein up to 1% w/w sesquisuccinate complex is added to facilitate crystal growth.

20. The method of claim 1 wherein said compound of formula I is selected from the group consisting of crystal form A, crystal form B, crystal form C, crystal form D, crystal form E, crystal form F, crystal form G, crystal form H, amorphous form and mixtures thereof.

21. The method of claim 20 wherein said compound of formula I is crystal form A.
